(19) United States
(12) Patent Application Publication
(10) Pub. No.: US 2004/0186559 A1
(43) Pub. Date: Sep. 23, 2004

(54) Vascular prosthesis

(76) Inventor: Eric Perouse, Paris (FR)

Correspondence Address: WENDEROTH, LIND & PONACK, L.L.P., 2033 K STREET N. W., SUITE 800, WASHINGTON, DC, 20006-1021, US

(21) Appl. No.: 10/782,898

(22) Filed: Feb. 23, 2004

(30) Foreign Application Priority Data
Mar. 17, 2003 (FR) 03 03 260

(51) Int. Cl. A61F 2/06

(52) U.S. Cl. 623/1.31; 623/1.36

(57) ABSTRACT

The vascular prosthesis comprises a flexible duct that is elastically deformable lengthwise. It further comprises adjustable means for fixing the spacing between two distant sections of the length of the flexible duct, the duct extending beyond at least one of the first and second sections so as to form at least one segment that is elastically deformable lengthwise.

The present invention relates to a vascular prosthesis of the type comprising a flexible duct that is elastically deformable lengthwise.

### BACKGROUND OF THE INVENTION

Nowadays, it is known to use vascular prostheses to replace a damaged segment of vein or artery. Such a prosthesis comprises a flexible duct that is elastically deformable lengthwise and that defines a circuit for blood flow. Each of the ends of the prosthesis is secured to the remaining segment of the damaged artery or vein.

The length of the vascular prosthesis must be matched to the length of the segment that is to be replaced. The prosthesis must be put into place and fixed at each of its ends under a certain amount of tension.

When the ends of the vascular prosthesis are connected by sutures, the surgeon performs anastomosis at a first end of the prosthesis and then pulls the prosthesis so as to tension it. The surgeon then cuts the prosthesis to length prior to performing anastomosis at its other end.

Such determination of length by cutting off one end of the prosthesis can be performed only when the prosthesis is secured by stitches or sutures.

Nevertheless, it is nowadays a known practice to put such prostheses into place by colioscopy. The prosthesis is then fitted with fastener devices such as agrafes at each of its ends. It is then no longer possible during the operation to cut off one end of the prosthesis in order to determine its length. It is therefore necessary for the prosthesis to have the right length initially.

However before beginning the operation, the surgeon cannot foresee how long the prosthesis will need to be, so, when performing an operation, the surgeon needs to have available a series of prostheses of different lengths. Thus, insofar as each prosthesis is relatively expensive, the need to have a complete series of prostheses available makes this type of operation very expensive.

### OBJECTS AND SUMMARY OF THE INVENTION

An object of the invention is to propose a vascular prosthesis enabling the cost of such an operation to be reduced.

To this end, the invention provides a vascular prosthesis of the above-specified type, comprising adjustable means for fixing the spacing between two distant sections of the length of the flexible duct, the duct extending beyond at least one of the first and second sections so as to form at least one segment that is elastically deformable lengthwise.

In particular embodiments of the invention, the vascular prosthesis further comprises one or more of the following features:

the adjustable means for fixing the spacing comprise a spacer having one end secured to the first section of the flexible duct and having an opposite end presenting means for securing to a second section that is selected along the length of the duct;

the flexible duct is engaged around the spacer between the first and second sections;

the retention means comprise a ring engaged around the flexible duct, the ring and the spacer having complementary resilient engagement means suitable for securing the ring to the second end of the spacer with the flexible duct being clamped and retained between the ring and the spacer;

the spacer comprises a hollow tube of diameter substantially equal to the inside diameter of said flexible duct;

the first section of the flexible duct is sewn to the first end of the spacer;

the hollow tube includes a flared portion at its first end;

the flexible duct is corrugated; and

the flexible duct includes fasteners preinstalled at each of its ends.

### BRIEF DESCRIPTION OF THE DRAWINGS

The invention will be better understood on reading the following description given purely by way of example and made with reference to the accompanying drawings, in which:

FIG. 1 is a longitudinal section view of a vascular prosthesis of the invention implanted in an organism; and

FIG. 2 is a longitudinal section view of a prosthesis of the invention prior to being implanted.

### MORE DETAILED DESCRIPTION

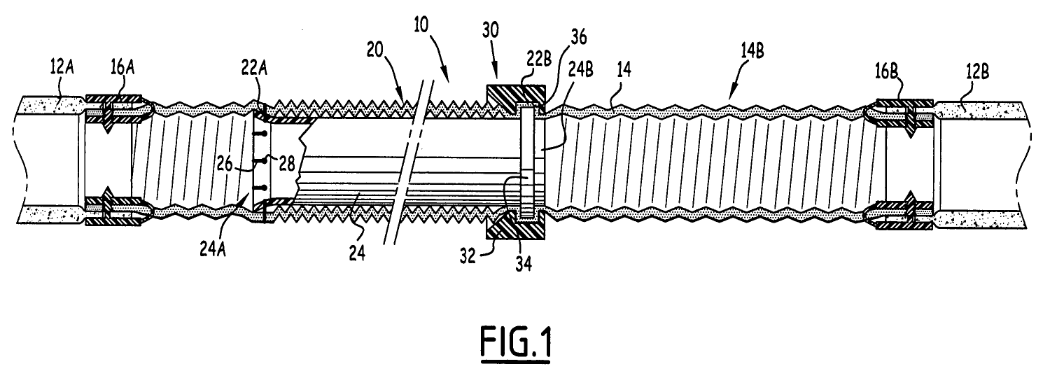

FIG. 1 shows a vascular prosthesis 10 of the invention implanted to replace a damaged segment of artery. Thus, the vascular prosthesis 10 is connected to two end segments 12A, 12B of the same artery from which a middle portion has been removed.

The vascular prosthesis 10 comprises a flexible tubular duct 14. This duct is formed by a woven or knitted cylindrical sheath. This duct is elastically deformable lengthwise. For this purpose, the duct is corrugated, i.e. over the major portion of its length it presents permanent ridge-and-furrow deformations enabling the duct to be lengthened or shortened. The duct then behaves overall like a bellows. This type of permanent deformation in the surface of a tubular duct can also be referred to as being “concertina-like”.

The length of the duct 14 as a function of the extent to which it is stretched lies in the range 10 centimeters (cm) to 80 cm, and its diameter lies in the range 0.5 cm to 4 cm.

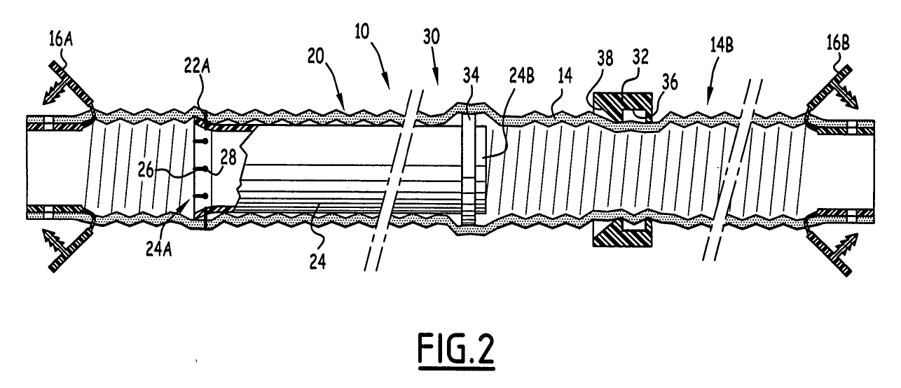

At each of its ends, the flexible duct 14 is connected to the corresponding segment 12A, 12B by means of agrafes 16A, 16B distributed around the periphery of each end. The agrafes can be of any appropriate type. They are initially pre-installed at the ends of the flexible duct 14 prior to the prosthesis being put into place, as can be seen in FIG. 2.

According to the invention, the vascular prosthesis includes adjustable means 20 for fixing the spacing between two distinct sections 22A, 22B of the flexible duct 14.

More precisely, and as shown in the figures, these means 20 comprise a spacer 24 having a first end 24A thereof permanently connected to a first section 22A of the duct. A second section 22B selected depending on the length of the duct is secured to the second end 24B.

This spacer 24 is constituted by a rectilinear tube of determined length. The tube may be flexible or rigid. The diameter of the tube 24 is substantially equal to the diameter of the duct 14. The length of the tube lies in the range 5 cm to 20 cm. The tube 24 is disposed inside the duct 14.

At its first end 24A, the tube 24 presents a flared portion such that its diameter increases progressively towards said first end. The duct 14 is secured to said first end, e.g. by stitches, using a strand 26 engaged in holes 28 formed through the flared end 24A of the duct. The section 22A of the duct sewn to the end 24A extends, at a short distance from the end of the duct where the agrafes 16A are secured, where said short distance is of centimeter order, for example.

The means 20 for fixing the spacing between the two sections comprise, at the second end 24B of the spacer, adjustable means 30 for securing to the second section 22B of the duct 14. These means 30 comprise a ring 32 engaged around the duct 14.

The ring 32 and the tube 24 comprise complementary resilient engagement means serving, once engaged, to prevent the ring 32 at the end 24B of the tube from moving relative to the duct 14 that is clamped and held via its second section 22B.

More precisely, and as shown in the figures, the tube 24 presents an outer peripheral bead 34 at its second end 24B. On the inside, the ring 32 has a peripheral groove 36 for receiving the bead 34. In front of the groove 36, on its side facing towards the bead 34, and prior to resilient engagement, the ring 32 presents a peripheral chamfer 38 defining a frustoconical surface that makes it easier to engage the ring on the bead 34.

Prior to being put into place, the vascular prosthesis is as shown in FIG. 2. The duct 14 is connected to the tube 24 at its end 24A. The ring 32 is engaged around the free segment referenced 14B beyond the end 24B of the duct.

While it is being put into place, the end fitted with agrafes 16A is initially secured to the segment 12A by putting the agrafes into place.

The total length of the prosthesis is then adjusted by the practitioner as a function of the distance between the two segments 12A, 12B. For this purpose, an appropriate length of the duct 14 is engaged on and stored around the tube 24 so that the free segment 14B can be maintained with the desired amount of tension. The ring 32 is then engaged on the end 24B of the tube so as to prevent the section 22B of the tube held under the ring from moving.

The agrafes 16B are then tightened so as to secure the duct 14 to the segment 12B.

It will be understood that prior to the retaining ring 32 being put into place, the length of duct 14 engaged around the tube 24 can be adjusted so as to adjust the total length of the vascular prosthesis.

After the ring 32 has been put into place, the free segment 14B of the duct is maintained under satisfactory tension, as a function of the length of duct that is retained on the tube 24.

Thus, the length of the vascular prosthesis can be adjusted while allowing the surgeon to obtain satisfactory tensioning of the prosthesis.

In addition, there is no need to cut the duct 14, and the vascular prosthesis can be secured by agrafes that have been preinstalled at each of its ends.

Such an arrangement can also be implemented on a branch of a bifurcated vascular prosthesis, i.e. a prosthesis that is generally Y-shaped.

What is claimed is:

1. A vascular prosthesis comprising a flexible duct that is elastically deformable lengthwise, the prosthesis including adjustable means for fixing the spacing between two distant sections of the length of the flexible duct, the duct extending beyond at least one of the first and second sections so as to form at least one segment that is elastically deformable lengthwise.

2. A vascular prosthesis according to claim 1, wherein the adjustable means for fixing the spacing comprise a spacer having one end secured to the first section of the flexible duct and having an opposite end presenting means for securing to a second section that is selected along the length of the duct.

3. A vascular prosthesis according to claim 2, wherein the flexible duct is engaged around the spacer between the first and second sections.

4. A vascular prosthesis according to claim 3, wherein the retention means comprise a ring engaged around the flexible duct, the ring and the spacer having complementary resilient engagement means suitable for securing the ring to the second end of the spacer with the flexible duct being clamped and retained between the ring and the spacer.

5. A vascular prosthesis according to claim 2, wherein the spacer comprises a hollow tube of diameter substantially equal to the inside diameter of said flexible duct.

6. A vascular prosthesis according to claim 2, wherein the first section of the flexible duct is sewn to the first end of the spacer.

7. A vascular prosthesis according to claim 6, wherein the hollow tube includes a flared portion at its first end.

8. A vascular prosthesis according to claim 1, wherein the flexible duct is corrugated.

9. A vascular prosthesis according to claim 1, wherein the flexible duct includes fasteners preinstalled at each of its ends.
